(19) United States
(12) Patent Application Publication
(10) Pub. No.: US 2024/0325605 A1
(43) Pub. Date: Oct. 3, 2024

(54) Tissue Scaffold with Patterned Microstructure

(71) Applicant: BVW Holding AG, Cham (CH)

(72) Inventors: Lukas Bluecher, Eurasberg (DE); Michael Milbocker, Holliston, MA (US)

(21) Appl. No.: 18/623,287

(22) Filed: Apr. 1, 2024

Related U.S. Application Data
(60) Provisional application No. 63/456,710, filed on Apr. 3, 2023.

(51) Int. Cl. A61L 27/50; A61L 27/36; A61L 27/38; G03F 7/20

(52) U.S. Cl. CPC A61L 27/50; A61L 27/3633; A61L 27/3886; A61L 2420/02; G03F 7/2002

(57) ABSTRACT

Provided herein is a tissue scaffold that may include microstructure patterns on one or more surfaces that can modify the physical properties of the tissue scaffold. The microstructure patterns may be cell-directing across a larger spatial range than the prior art which uses chemically modified substrates. The present disclosure further includes tissue scaffold configured such that cells may infiltrate the tissue scaffold by responding to patterns of surface energy gradients on the tissue scaffold and the cells may not be constrained into patterns by physical means. As the cells proliferate, they associate and orient in response to long-range patterns to form confluent monolayers of cells while constructing functional macro-structure across the tissue scaffold surface.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application claims benefit of the following patent application(s) which is/are hereby incorporated by reference: 63/456,710 filed on Apr. 3, 2023.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present invention relates generally to tissue and cellular scaffolds having microstructure surfaces associated therewith.

Living cells and tissues can display high sensitivity to local micro- and nanoscale chemical and topographic patterns, including those provided in vivo by complex and well-defined structures of extracellular matrix (ECM). However, given the small scale of the underlying interactions, their effect on cell and tissue function are far from completely understood.

Studies of engineered cell-biomaterial interactions at the subcellular, nanoscale level provide some evidence for potential importance of sub-micrometer cues for cell signaling, adhesion, growth, and differentiation. However, initial attempts based on short-range stimuli to direct functional tissue formation have frequently not been bioinspired or biomimetic and have failed to reproduce the multiscale long-range effects of complex ECM structures and associated chemical ligands, which control integrated multicellular ensembles on scales ranging from a few micrometers to hundreds of micrometers. Recent advances in nanofabrication techniques can enable the design and fabrication of scalable scaffolding materials mimicking the structural and mechanical cues present in the in vivo ECM environment.

Tissue formation, wound repair, and many disease processes depend on expression and cell-mediated assembly of the appropriate ECM proteins. ECM provides a long-range organizing structure necessary for cell-mediated assembly of functional tissue. This long-range structure can be interpreted as a type of cellular language recognized by appropriate cell types to organize into biologically functional macroscopic structures.

Cell signaling using various cytokines are currently used to promote cell-specific infiltration onto synthetic tissue scaffolds. However, these cytokines typically provide only a localized signal to cells. While cytokines may promote infiltration of particular cell types, they provide no information on how these cells should organize macroscopically within cell types and between cell types. Long-range cell signaling may be generally lacking in present day tissue scaffolds.

Macroscopic or long-range cellular organization and resulting tissue may be defined as any biologically functional tissue of size at least 10 times the size of the constituent cells making up the macroscopic tissue structure.

The importance of long-range cell signaling may be illustrated by the ECM. Orientation of ECM fibers may be essential for normal tissue development and homeostasis. The organization of the ECM can, however, go awry in many diseases and at sites of injury producing the unaligned collagen fibers that form in scar tissue. Scar tissue typically lacks the biological functionality of the tissue it replaces.

As such, a goal of regenerative medicine may be to promote formation of new tissue that closely resembles the normal tissue (as compared to damaged or diseased tissue) in organization and function. Controlling cell growth in a long-range spatially defined way enables regeneration of damaged or diseased tissues having the proper orientation of constituent cells and/or orientation of molecular complexes that the cells produce. Typically, these long-range spatially organizing signals are generated by surface energy gradients, often mediated by patterns of hydrophilic and hydrophobic loci.

In particular, surface energy gradients direct the arrangement of ECM fibrils to correspond to cellular actin filaments by using cell surface receptors that are indirectly connected to the actin cytoskeleton. Therefore, a major challenge in regenerative medicine is to promote cells to assemble ECM fibrils, such as collagen, into particular long-range orientations or alignments on a scaffold device in order to generate tissues with the required functional properties.

Muscle tissue possesses complex structural organization on multiple scales, from micro-(1 to 100 microns) to macro-(100 to several 1000 microns) scales, but macro-scale control of function has not been extensively analyzed. For example, myocardium may be an ensemble of different cell types embedded in the complex and well-defined structures of the ECM and arranged on macro-scale topographical and molecular patterns. Although the structure of cardiac tissue is highly organized in vivo, cardiomyocyte ensembles lose their native organization and adopt random distribution when cultured in vitro by common culturing techniques, potentially compromising many of their physiological properties.

A variety of methods such as mechanical stretching, microcontact printing, and electrical stimulation have been used to engineer better organized cardiomyocyte cultures. Both 2D and 3D substrata with 10 μm feature size have been employed to direct cardiomyocytes into anisotropic arrangements for electrophysiological and mechanical characterization. However, it may be likely that the structure and function of the in vivo cardiac tissue are regulated by much larger, macroscale cues provided by the ECM, which might exercise complex, multiscale control of cell and tissue function. Thus, it is important to investigate and understand whether patterned control over the cell-material interface on the macro-scale facilitates the creation of truly biomimetic cardiac tissue constructs that recapitulate the structural and functional aspects of the in vivo large scale tissue phenotype.

In addition, the ability to generate uniformly controlled (both structurally and functionally) and precisely defined engineered biologic tissue that is robust and reproducible will likely be necessary for truly regenerative tissue scaffolds. In this regard, the inability to direct the long-range differentiation of multipotent progenitors specifically to mature muscle cells remains a major obstacle for optimal in vivo cellular genesis during soft tissue repair following injury.

Furthermore, while methods of cell-based therapy using cells on scaffolds exist, their use may be of limited benefit to the extent they do not sufficiently support long-range growth, differentiation and function of cells for a functional engineered biologic tissue.

What is needed, then, is a scaffold device that yields in some aspects long-range growth, differentiation, and function of cells for a functional engineered biological tissue that better represents biological tissue.

### BRIEF SUMMARY

The present disclosure is based, at least in part, on the discovery that the surface energy of microstructures can generate cell-adhesive patterns in nano- and micro-scale dimensions using a technique for surface-modifying solids or polymers to generate devices that have utility as biological scaffold materials. These scaffold materials can be used to generate functional tissue ex vivo to be implanted, or the scaffold materials can be implanted directly. Scaffold materials which are implanted directly can direct functional tissue repair or be used to create tissue compatible interfaces between living tissue and nonliving implants, such as prosthetics, sensors, electrodes, and the like.

Some embodiments, as disclosed herein, may generate a cellular language which can be used, for example, but not limited to, in regenerative medicine, wound repair, and/or transplant biology. Further, said embodiments may be used in screening assays to determine the effects of a test compound on living tissue by examining the effect of the test compound on various biological responses, such as for example, but not limited to, cell viability, cell growth, migration, differentiation and maintenance of cell phenotype.

Accordingly, in one aspect, some embodiments may provide micro- and/or nano-structured surfaces for scaffolds for tissue generation, both in vivo and ex vivo. In one embodiment, the tissue scaffold may comprise a base layer comprising a first pattern of microstructures, and a capping layer comprising a smooth layer or may include a second pattern of microstructures.

In another embodiment, a tissue scaffold may comprise a base layer comprising a pattern of microstructures; and a capping layer on each microstructure comprising one of a hydrophilic and hydrophobic layer. The hydrophobicity or surface energy of the capping layer may be due to the chemical composition of the capping substance, the geometry of the capping layer, or both.

In another embodiment, the tissue scaffold may comprise a base layer comprising a pattern of microstructures, a capping layer comprising a hydrophilic or hydrophobic layer, wherein the hydrophilic layer has a contact angle with water that may be equal to or below 100° and the hydrophobic layer may have a contact angle with water above 100°. More generally, the difference between the hydrophobic angle and the hydrophilic angles may be at least 5 degrees. The capping layers may comprise a hydrophobic/hydrophilic material or a single material formed as a microstructure with the desired contact angle, or one or more of each capping layer types.

In another embodiment, the tissue scaffold may include a base layer comprising a pattern of microstructures, a capping layer comprising a hydrophilic and/or hydrophobic layer, wherein if the hydrophilic layer is present, said layer has a contact angle with water that may be equal to or below 100° and if the hydrophobic layer is present, said layer has a contact angle with water above 100°. More generally, the difference between the hydrophobic angle and the hydrophilic angles may be at least 10 degrees, wherein, the microstructures are arranged in zones comprising a fixed pattern of hydrophilic and hydrophobic end caps.

In certain embodiments, the microstructures may be arranged in a square grid or triangular grid on the substrate surface. In some embodiments, the zones of microstructures may include blocks of microstructures that may be hexagonal, triangular, or quadrangular, and their three-dimensional analogues.

In another embodiment, an artificial tissue may include living cells attached to a tissue scaffold wherein the scaffold may comprise a base layer having a pattern of microstructures disposed thereon. The base layer may further include a capping layer comprising a pattern of microstructures or materials with at least two contact angles.

In another embodiment, an artificial tissue may include living cells attached to a tissue scaffold wherein the scaffold may comprise a base layer having a pattern of microstructures disposed thereon. The scaffold may further comprise a capping layer comprising a pattern of microstructures or a material with at least two contact angles wherein the combination of the base layer and capping layer form a Wenzel-Cassie interface when brought into contact with a liquid.

In embodiments where the liquid is a compound liquid, the liquid components may separate into discretely separate Wenzel zones. These Wenzel zones may be connected by capillary bridges. The capillary bridges comprising one component of the compound liquid may be interwoven with the capillary bridges comprising another component of the compound liquid.

In another embodiment, an artificial tissue may comprise living cells attached to a tissue scaffold that may include a base layer having a pattern of microstructures disposed thereon and further including a capping layer comprising a pattern of microstructures or a material with at least two contact angles wherein the combination of the base layer, pillars and capping layer form a Wenzel-Cassie interface when brought into contact with a liquid. When the liquid is a compound liquid, the liquid components may separate into Wenzel zones. And when the compound liquid resides between said artificial tissue and a living tissue, the Wenzel zones may form adhesive capillary bridges to said living tissue.

In some embodiments disclosed herein, the patterned tissue scaffold may be composed of polymer substrate for use in the compositions and methods to generate tissue engineered soft tissue as disclosed herein. The microstructures on the patterned tissue scaffold may be spatially organized from the nanometer to centimeter length scales and can be generated via methods described herein.

In certain embodiments, the polymers may include, for example, but not limited to, biopolymer (e.g., protein, carbohydrate, glycoprotein etc.,) and can be deposited onto a transitional polymer surface using patterning techniques that allow for nanometer scale spatial positioning of the deposited polymers.

The patterning techniques disclosed herein may include, but are not limited to, soft-lithography, self-assembly, vapor deposition and photolithography. Once on the surface, inter-polymer interactions may attract the polymers together such that they may become bound together. These interactions may be hydrophilic, hydrophobic, ionic, covalent, Van der Waals, hydrogen bonding and/or physical entanglement depending on the specific polymers involved.

In another embodiment, an artificial tissue including living cells that may be attached to a tissue scaffold comprising a base layer such that the base lay may include a pattern of microstructures and a capping layer comprising a pattern of microstructures or a material with at least two contact angles. The combination of the base layer having microstructures and capping layer may form a Wenzel-Cassie interface when brought into contact with a liquid. Further, the pillars may be arranged in a pattern creating a region of regions of hydrophilic/hydrophobic surface energies and the region or regions may generate a long-range structure. When the liquid is a compound liquid, the liquid components may separate into Wenzel zones. And when the compound liquid resides between said artificial tissue and living tissue the Wenzel zones may form adhesive capillary bridges to said living tissue.

In another embodiment, a tissue scaffold may be formed to include a pattern of microstructures on a base layer by photolithography to form a substrate having a patterned base layer. The scaffold may further include a capping layer by depositing the capping layer onto the patterned base layer to form a substrate having a patterned or hydrophilic/hydrophobic capping layer. The patterned capping layer may be contacted with a tissue layer to generate a substrate having a patterned tissue adhesive layer.

In another embodiment, a method may include providing a tissue scaffold comprising a pattern of microstructure on a base layer by photolithography to form a substrate having a patterned base layer; depositing a capping layer onto the patterned base layer to form a substrate having a patterned or hydrophilic/hydrophobic capping layer; contacting the substrate having the patterned tissue adhesive layer with cells and culturing under conditions suitable for the production of extracellular matrix components; and removing the cells from the substrate to provide a biological tissue scaffold comprising an extracellular matrix component.

In another embodiment, a method of using a tissue scaffold may include a pattern of pillars on a base layer comprising a substrate having a patterned base layer; depositing a capping layer onto the patterned base layer to form a substrate having a patterned or hydrophilic/hydrophobic capping layer; contacting the substrate having the patterned tissue adhesive layer with cells and culturing under conditions suitable for the production of extracellular matrix components; and then contacting said tissue scaffold with extracellular matrix components with living tissue.

In another embodiment, a method for identifying a compound that modulates a tissue function may include providing a patterned scaffold for tissue as described herein, contacting the tissue with a test compound, and measuring the effect of the test compound on a tissue function in the presence and absence of the test compound, wherein a modulation of the tissue function in the presence of the test compound as compared to the tissue function in the absence of the test compound indicates that the test compound modulates a tissue function, thereby identifying a compound that modulates a tissue function.

In another embodiment, a method for identifying a long-range microstructure pattern that modulates a tissue function may include providing a patterned scaffold for tissue as described herein comprising patterned regions that may be arranged in a long-range microstructure pattern, contacting the tissue with a test long-range microstructure pattern, and measuring the effect of the test long-range microstructure pattern on a tissue function in the presence and absence of the test long-range microstructure pattern, wherein a modulation of the tissue function in the presence of the test long-range microstructure pattern as compared to the tissue function in the absence of the test long-range microstructure pattern indicates that the test long-range microstructure pattern modulates a tissue function, thereby identifying a long-range microstructure pattern that modulates a tissue function.

In another embodiment, methods for identifying a long-range microstructure pattern useful for treating a tissue defect are disclosed wherein the methods include providing a patterned scaffold for tissue as described herein, contacting the tissue with a test long-range microstructure pattern, and measuring the effect of the test long-range microstructure pattern on a tissue function in the presence and absence of the test long-range microstructure pattern, wherein a modulation of the tissue function in the presence of the test long-range microstructure pattern as compared to the tissue function in the absence of the test long-range microstructure pattern indicates that the test long-range microstructure pattern modulates a tissue function, thereby identifying a long-range microstructure pattern useful for treating or preventing a tissue disease.

In another embodiment, a tissue scaffold is disclosed wherein the scaffold may be used for treating a tissue defect.

### BRIEF DESCRIPTION OF THE SEVERAL VIEWS OF THE DRAWINGS

FIG. 1 is an illustration of an embodiment of a tissue scaffold as disclosed herein.

FIGS. 2A-2D are illustrations of coded zones of microstructures as disclosed herein.

FIGS. 3A-3D are illustrations of coded regions of microstructures which may act as cell-communicating regions as disclosed herein.

### DETAILED DESCRIPTION

The present disclosure is based, at least in part, on the discovery of cell signaling long-range patterns in nano- and micro-scale dimensions using a technique for surface-modifying solids or polymers to prepare devices that have utility as tissue scaffold materials. The patterned surface modifications generate surface energy gradients when a tissue scaffold comprising said long-range patterns comes into contact with living tissue or cells within a culture medium.

The phrase “contact angle” may be understood to be a quantitative measure of wetting of a solid by a liquid. The contact angle may be geometrically defined as the angle formed by a liquid drop at the three-phase boundary where a liquid, gas, and solid intersect after the liquid drop has been in contact with the gas and solid at 20° C. for a given amount of time, being at least one (1) second. Unless stated otherwise, in certain embodiments the gas may be air and the liquid may be water.

The phrases “compound fluid” and “compound liquid” may be understood to refer to a liquid composed of at least two substances in liquid form. For example, a mixture of water and alcohol may be a compound liquid referred to as a binary solution.

The phrase “long-range pattern” may be understood to refer to any pattern of microstructure features with a spatial periodicity greater than the dimensions of the microstructure. Typically, a Fourier transform of the long-range microstructure pattern may be understood to indicate multiple periodicities.

The term “resorbable” may be understood to refer to polymers that may undergo absorption into the circulation of cells or tissue when implanted.

As used herein, the term “depositing” may be understood to refer to a process of placing or applying an item or substance onto another item or substance (which may be identical to, similar to, or dissimilar to the first item or substance). Depositing may include, but is not limited to, methods of using spraying, dip casting, spin coating, evaporative methods, sputter methods, immersion methods, extractive deposition methods, or other methods to associate the items or substances. The term depositing may include methods of applying the item or substance to substantially the entire surface as well as applying the item or substance to a portion of the surface.

As used herein, the term “continuous layer” may be understood to be refer to a layer that may be formed by a matrix of individual molecules that are chemically bonded or mechanically linked to each other.

As used herein, the various forms of the term “modulate” are intended to include stimulation (e.g., increasing or upregulating a particular response or activity) and inhibition (e.g., decreasing or down regulating a particular response or activity).

As used herein, the term “contacting” (e.g., contacting a tissue with a test compound) may be understood to be intended to include any form of interaction (e.g., direct or indirect interaction) of a test compound and a tissue. The term contacting includes incubating a compound and a tissue (e.g., adding the test compound to a tissue).

As used herein, the term “comprising” may be understood to refer to include the recited elements as well as other elements which can also be present though not specifically stated. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the disclosure, yet open to the inclusion of unspecified elements, essential or not (“comprising”).

As used herein, the term “code” may be understood to refer to a microstructure comprising a juxtaposition of surface energy gradients in a defined surface area. In some embodiments, the surface energy gradients may be defined by microstructure surfaces of different surface energy. For example, a low surface energy (hydrophobic) microstructure may be juxtaposed with a high surface energy (hydrophilic) microstructure. Labeling these microstructures as 0 for a hydrophobic microstructures and as 1 for a hydrophilic microstructures, then an array of 0's and 1's may be used to describe a code. The array can be two dimensional, or may be higher dimensional when two-dimensional microstructured surfaces are stacked, for example, but not limited to hierarchical.

Considering a two-dimensional code, the zone defining the code may be comprised of lines of 0's and 1's. Considering a first line as an object, then the adjacent line can be considered a permutation of the first line. Consequently, a code may be specified as a first line of 0's and 1's and a permutation group acting on a specified first line. Selecting different first lines will generate different zones of code for the same group operation. Thus the zones specified by a code may represent the elements of a mathematical group.

As used herein, the term “substrate” may be understood to refer to any suitable carrier material. In some embodiments, the carrier material may be a material to which the cells are able to attach or adhere in order to form the corresponding cell composite, e.g. a tissue engineered smooth muscle composition. In some embodiments, the matrix or carrier material, respectively, may be present already in a three-dimensional form desired for later application. For example, bovine pericardial tissue can be used as a matrix which may be crosslinked with collagen, decellularized and photo-fixed.

As used herein, the term “pattern” may be understood to refer to mean a pre-determined arrangement or design, generally a substantially microscale design of coded microstructures in a surface as described herein.

As disclosed herein, the inventors have discovered that a long-range patterned polymer platform, referred to herein as a “coded microstructured scaffold” or “CMS”, fabricated to comprise an array of coded zones can organize immature cells, for example, myocytes, in an anisotropic manner, and the CMS may promote mature phenotypes in anisotropically arranged tissue relative to the same cells cultured on a substrate lacking the coded zones. Accordingly, the inventors have discovered a microstructure code may produce functional tissue engineered cells which may be capable of large scale functionality and have vastly superior function as compared to existing tissue engineered tissues of the prior art.

The devices and methods of the present disclosure as compared to previously described methods and devices of the prior art may be found to induce cell signaling that may cause cells to follow and organize into macro-scale structures defined by long-range energy gradients generated on the surface of a tissue scaffold. These long-range energy gradients may mimic an in vivo environment to properly concentrate and orient cell types to form a functional tissue.

Accordingly, the present disclosure may be a synthetic tissue scaffold wherein all or some portion of which may be implanted in living tissue to repair a soft tissue defect. The resulting tissue repair may be characterized by appropriate cellular organization and biological activity.

Certain embodiments disclosed herein may not rely upon short-range chemical treatment of the tissue scaffold. For example, avoiding the use of biologic cell-adhesive molecules to facilitate cell infiltration on the tissue scaffold may help prevent encumbrance of cell receptors that may be involved in natural cell spreading and extracellular interactions affecting cellular organization and tissue alignment.

In particular, embodiments disclosed herein may include a tissue scaffold which uses microstructure patterns to modify the physical properties of the substrate. Said embodiments may be more durable than previously described methods and devices that rely on treatment of the tissue scaffold surface with unstable, environmentally sensitive biofunctional molecules.

In some embodiments disclosed herein, the microstructure patterns may be cell-directing across a larger spatial range than the prior art which uses chemically modified substrates. One aspect may be that cells infiltrate the tissue scaffold by responding to patterns of surface energy gradients on the tissue scaffold as disclosed herein and the cells are not constrained into patterns by physical means. As the cells proliferate, they associate and orient in response to long-range patterns to form confluent monolayers of cells while constructing functional macro-structure across the tissue scaffold surface.

In some embodiments, the CMS as disclosed herein can be used to generate functional muscle tissue, e.g., functional engineered myocardium, as the CMS may be patterned, so that the cellular environment at multiple spatial scales may be modified in order to direct the maturation of in vitro differentiated cells, and to subsequently organize the in vitro differentiated cells into two-dimensional and three-dimensional tissue structures. In some embodiments, the CMS can be coated with agents, such as differentiation factors, which promote the differentiation of cells.

Some embodiments disclosed herein may have aspects that that represent a key advancement in the strategy for large-scale engineering of functional tissue from a stem cell source. In particular, one key aspect may be the ability to mature in vitro-differentiated cells into a functional tissue, as prior efforts to produce mature cells from precursors or progenitors is highly inefficient and does not produce functional tissue resembling mature adult tissue in vivo. In some instances, this may be because functional tissue is an ensemble of different cell types embedded in the complex structures of the extracellular matrix (ECM) arranged in topographical and chemical patterns.

In some embodiments, the patterns may be characterized by combinations of surface energy gradients. The code of the CMA in some embodiments may be a spatial distribution of surface energy gradients. In common culturing techniques this code may be absent and the resulting in vitro-differentiated cells lack their native organization, resulting in random distribution when cultured in vitro, compromising many of their physiological properties.

The cellular environment of the CMS in some embodiments may be engineered on the micrometer scale to encompass lengths of macroscopic length patterned zones, providing precise micro and long-range cues to allow the organization of differentiated cells to adopt a more mature organization. The patterned zones may be comprised on microstructures with a plurality of surface energy gradients. In an aqueous environment, these surface energy gradients may be manifested as hydrophilic and/or hydrophobic loci. Accordingly, a patterned zone or region may be comprised of microstructure corresponding to a particular combination of hydrophilic and hydrophobic loci. These zones or regions may in turn be arranged in a pattern to provide a long range structure. Structural features within the zones or regions may be scaled up in the combination of zones or regions to constructure self-similar patterns.

In some embodiments, factors that are engineered or manipulated to provide one or more benefits may include, but are not limited to, material mechanical properties, material solubility, spatial patterning of the topological features, e.g., ridges and pillars, soluble bioactive compounds, mechanical perturbation (cyclical or static strain, stress, shear, etc.), electrical stimulation, and/or thermal perturbation.

Accordingly, as described herein, methods and devices may be used in a broad range of applications, for example, in regenerative medicine, wound repair, transplant biology, drug delivery, testing the effect of substances upon cells, tissue formation, cell actuation, and developmental biology.

Accordingly, in one aspect, the present disclosure provides embodiments having patterned scaffolds for tissue. In one embodiment, a tissue scaffold may include a base layer comprising a pattern of microstructures. The base layer may further include a capping layer comprising a chemical surface energy modifying composition or a second surface energy modifying microstructure pattern.

In another embodiment, the tissue scaffold may comprise a base layer comprising a pattern of microstructures and a capping layer microstructure on each base layer microstructure comprising one of a hydrophilic and hydrophobic substance, wherein first and second microstructures form a Wenzel-Cassie interface when placed in contact with a wet target surface. The Wenzel-Cassie interface may generate a non-biologic cell adhesive layer disposed on the tissue scaffold.

In some embodiments disclosed herein, a base layer may be a solid, rigid, or hard polymeric surface, a semi-rigid polymeric surface, a soft polymeric surface, a hard non-polymeric surface, a semi-rigid non-polymeric surface, or a soft non-polymeric surface, or combinations thereof.

In some embodiments, a base layer may be biologically absorbable and/or chemically inert.

In some embodiments, a base layer may include one or more surfaces or components arranged hierarchically. For example, a base layer may comprise soft polymeric pillars including a top surface on each pillar that may be populated with smaller pillars. With such a base layer, a surface energy gradient may exist between base pillars and which may be different from the surface gradient between capping layer pillars.

In some embodiments, a base layer for use in the compositions and methods of the disclosure may have a Young's modulus of about 0.000001-0.01, 0.005-0.2, 0.005-0.5, 0.05-1.0, 0.075-1.0, 0.1-2.0, 1.0-2.0, 1.5-5.0, 2.0-5.0, 3.0-7.0, 3.0-10, 5.0-15, 5.0-20, 10-20, 15-30, 20-30, 25-50, 30-50, 50-75, 50-100, 75-125,100-150, 125-150, 150-200, 175-200, 200-250, or about 250-300 gigapascals (GPa).

Ranges and values intermediate to the above recited ranges and values are also contemplated to be part of the disclosure. For example, but not a limiting example, a Young's modulus of about 3.5-9.5, or 4.0-5.0 GPa may be intended to be encompassed by the present disclosure for application where minimal distortion of the microstructured pattern may be desired. However, the Young's modulus of natural tissue ranges from about 1 to 1000 kilopascals (kPa). Like the example provided above, other examples are also contemplated of other ranges for each of the values recited above in the preceding paragraph.

Embodiments of the present disclosure intended to repair dynamical soft tissue, for example, but not limited to, the heart, a Young's modulus of about 2.2-10.0 kPa or 4.0-5.0 kPa may be desired.

In other embodiments, a base layer for use in the compositions and methods of the disclosure may have a Young's modulus of about 0.001-0.1, 0.005-0.2, 0.005-0.5, 0.05-1.0, 0.075-1.0, 0.1-2.0, 1.0-2.0, 1.5-5.0, 2.0-5.0, 3.0-7.0, 3.0-10, 5.0-15, 5.0-20, 10-20, 15-30, 20-30, 25-50, 30-50, 50-75, 50-100, 75-125, 100-150, 125-150, 150-200, 175-200, 200-250, or about 250-300 kilopascals (kPa). Ranges and values intermediate to the above recited ranges and values are also contemplated to be part of the disclosure. For example, a Young's modulus of about 3.5-9.5, or 8.0-9.0 kPa may be intended to be encompassed by the present disclosure. Like the example provided above, other examples are also contemplated of other ranges for each of the values recited above in the paragraph.

In one embodiment, the base layer may be selected from the group consisting of a hard polymeric surface, a semi-rigid polymeric surface, and/or a soft polymeric surface, a hard non-polymeric surface, a semi-rigid non-polymeric surface, and/or a soft non-polymeric surface, and combinations thereof.

In one embodiment, the base layer may comprise a polyamide, a polyurethane, a polyurea, a polyester, a polyketone, a polyimide, a polysulfide, a polysulfoxide, a polysulfone, a polythiophene, a polypyridine, a polypyrrole, polyethers, silicone (polysiloxane), polysaccharides, fluoropolymers, epoxies, aramides, amides, imides, polypeptides, polyethylene, polystyrene, polypropylene, glass reinforced epoxies, liquid crystal polymers, thermoplastics, bismaleimide-triazine (BT) resins, benzocyclobutene ABFGx13, low coefficient of thermal expansion (CTE) films of glass and epoxies, polyvinyls, polyacrylics, polyacrylates, polycarbonate, polytetrafluoroethylene (PTFE), polyethylene terephthalate (PET), quartz, silicon (e.g., silicon wafers), glass, ceramic, metals and metal alloys including titanium, titanium alloys, tantalum, zirconium, stainless steel and cobalt-chromium alloys, metal oxides, poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, polyrethylene-co-vinyl acetate), poly(ethylene glycol), poly(methacrylic acid), polylactides (PLA), polyglycolides (PGA), poly(lactide-co-glycolides) (PLGA), polyanhydrides, polyphosphazenes, polygermanes, polyorthoesters, polyolefins, polycarbonates, biopolymers, such as a silk, collagen, copolymers and derivatives thereof, and composites including these polymers. In one preferred embodiment, the base layer is may be silicon, polyetheretherketone (PEEK), nylon, including nylon 6,6 or PET, and combinations thereof.

In some embodiments, a CMS may be a microstructure patterned polymer, e.g., a biopolymer, and can be created in certain embodiments by providing a transitional polymer on a substrate; depositing a biopolymer on the transitional polymer; shaping the polymer into a structure having a selected pattern on the transitional polymer (e.g., poly(N-Isopropylacrylamide); and releasing the biopolymer from the transitional polymer with the polymer's structure and integrity intact.

In some embodiments, the microstructure patterns and polymers may be based upon, e.g., extracellular matrix protein, a biologically active carbohydrate, a biologically derived homopolymer, silks, polyprotein (e.g., poly(lysine)) or a combination thereof. For example, the polymer can be selected from the group consisting of fibronectin, vitronectin, laminin, collagen, fibrinogen, silk or silk fibrin. The polymer component of the structure can comprise a combination of two or more ECM proteins such as fibronectin, vitronectin, laminin, collagens, fibrinogen and structurally related protein (e.g. fibrin).

In another embodiment, the base layer may be resorbable polylactic acid or poly(lactic-co-glycolic acid). In another embodiment, the base layer may be resorbable polyurethane. In another embodiment, the base layer may be non-resorbable polytetrafluoroethylene.

For certain embodiments, medical devices may be used that incorporate one or more embodiments disclosed herein, said medical devices may include, for example, but not limited to, diagnostic implant devices, biosensors, stimulators, neural stimulators, neural activity recorders, diabetic implants such as glucose monitoring devices, external fixation devices, external fixation implants, orthopedic trauma implants, implants for use in joint and spinal disorders/reconstruction such as plates, screws, rods, plugs, cages, scaffolds, artificial joints (e.g., hand, wrist, elbow, shoulder, spine, hip, knee, ankle), wires and the like, oncology related bone and soft tissue replacement devices, dental and oral/maxillofacial devices, cardiovascular implants such as stents, catheters, valves, rings, implantable defibrillators, and the like, contact lenses, ocular implants, keratoprosthesis, dermatologic implants, cosmetic implants, implantable medication delivery pumps; general surgery devices and implants such as but not limited to drainage catheters, shunts, tapes, meshes, ropes, cables, wires, sutures, skin staples, burn sheets, and vascular patches; and temporary/non-permanent implants.

The base layer of certain embodiments may comprise a pattern of microstructures having at least two microstructure elements wherein adjacent microstructure elements are separated by a space. In one embodiment, the width and spacing of the microstructure elements may be about 0.1 μm to about 1000 μm. In another embodiment, the microstructure elements are selected from pillars with a circular, square, triangular, hexagonal cross section. The sides of the pillars may be straight and perpendicular to the base layer surface or tapered distally and proximally. Pillars with a long dimension are ridges, pillars and the like.

The width and spacing of the pillars in some embodiments may be varied over the range from about 0.1 μm to about 1000 μm, from about 1 μm to about 500 μm, from about 1 μm to 250 μm, from about 5 μm to 250 μm, from about 1 μm to 100 μm, from about 1 μm to 90 μm, from about 1 μm to 80 μm, from about 1 μm to 70 μm, from about 1 μm to 60 μm, from about 1 μm to 50 μm, from about 1 μm to 40 μm, from about 1 μm to 30 μm, from about 1 μm to 20 μm, from about 1 μm to 10 μm, from about 2 μm to 100 μm, from about 2 μm to 90 μm, from about 2 μm to 80 μm, from about 2 μm to 70 μm, from about 2 μm to 60 μm, from about 2 μm to 50 μm, from about 2 μm to 40 μm, from about 2 μm to 30 μm, from about 2 μm to 20 μm, from about 2 m to m, from about 1 μm to 100 μm, from about 5 μm to about 160 μm, from about 5 μm to about 100 μm, from about 5 μm to about 90 μm, from about 5 μm to about 80 μm, from about 5 μm to about 70 μm, from about 5 μm to about 60 μm, from about 5 μm to about 50 μm, from about 5 μm to about 40 μm, from about 5 μm to about 30 μm, from about 5 μm to about 20 μm, and from about m to about 10 μm. Ranges and values intermediate to the above recited ranges and values are also contemplated to be part of the disclosure. As one example, a width and spacing of about 10-200, or 50-200 μm are intended to be encompassed by the present disclosure. Other examples are also contemplated by this disclosure based on the ranges and values recited above.

The width and spacing of the pillars can be equivalent or different. In the art, pitch of the pillars may be the sum of width and spacing. For example, both the width and spacing can be about 0.1, about 0.2, about 0.25, about 0.5, about 0.75, about 1, about 2, about 3, about 4, about 5, about 6, about 7, about 8, about 9, about 10, about 11, about 12, about 13, about 14, about 5, about 16, about 17, about 18, about 19, or about 20 μm. In other embodiments, the width can be about 0.1, about 0.2, about 0.25, about 0.5, about 0.75, about 1, about 2, about 3, about 4, about 5, about 6, about 7, about 8, about 9, about 10 μm, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 μm, and the spacing can be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 μm. Values and ranges intermediate to the above recited values are also contemplated to be part of the disclosure. For example, a width and spacing of about 5.0 and 30.0 are intended to be encompassed by the present disclosure. Other examples are also contemplated by this disclosure based on the ranges and values recited above.

In one embodiment, the pillars may be about 5 μm to 100 μm wide and spaced about 5 μm to 100 μm apart. In another embodiment, the pillars may be about 5 μm wide and spaced about 5 μm apart. In another embodiment, the pillars may be about 10 μm wide and spaced about 10 μm apart. In another embodiment, the pillars may be about 20 μm wide and spaced about 20 μm apart. In yet another embodiment, the pillars may be about 30 μm wide and spaced about 30 μm apart. In yet another embodiment, the pillars may be about 20 μm wide and spaced about 10 μm apart. Other examples are also contemplated by this disclosure based on the ranges and values recited above.

A capping substance for use in a capping layer of the tissue scaffold may include a moiety that when contacted with the patterned base layer forms a continuous layer on the pillars of the patterned base layer. In some embodiments, the capping substance may be a polymer. In some embodiments, the polymer may be formed from a PEG or silicone precursor. In some embodiments the polymer may be an alkoxide.

For embodiments having a polymeric alkoxide, the polymer may have from three to six alkoxide groups or a mixture of oxo and alkoxide groups. In certain embodiments, an alkoxide group may have from 2 to 4 carbon atoms, and may include, for example, ethoxide, propoxide, iso-propoxide, butoxide, iso-butoxide, tert-butoxide and fluorinated alkoxide.

In some embodiments described herein, the polymer of the capping layer may include cell adhesive moieties. Cell adhesive chemical compounds may be organic compounds comprising a phosphonic, carboxylic, sulfonic, phosphinic, phosphoric, sulfinic, or hydroxamic group. In some embodiment, the cell adhesive layer may comprise a phosphonate.

In certain embodiments, the tissue scaffolds may comprise living cells. It has been discovered that in some embodiments, cells may adhere to the patterned tissue scaffold in the absence of a cell adhesive layer disposed on the capping layer. Accordingly, in some embodiments, an artificial tissue may include living cells attached to a tissue scaffold comprising a base layer wherein the base layer may have a pattern of microstructures. In some embodiments, the base layer may include a capping layer comprising a second pattern of microstructures or capping layer of hydrophilic and hydrophobic polymers. In another embodiment, an artificial tissue may comprise living cells attached to a tissue scaffold comprising: a base layer comprising a pattern of microstructures and a capping layer comprising a long-range pattern of hydrophilic and hydrophobic caps.

The type of cells disclosed herein for some embodiments may include, for example, and is not limited to, fibroblasts, endothelial cells, keratinocytes, osteoblasts, chondroblasts and chondrocytes, hepatocytes, macrophages, cardiac muscle cells, smooth muscle cells, skeletal muscle cells, tendon cells, ligament cells, neural cells, epithelial cells, and stem cells. Stem cells may be understood to include, but are not limited to, embryonic stem cells, adult stem cells, and induced pluripotent stem cells. In one embodiment, the cells may be mesenchymal stem cells. In another embodiment, the cells may be human cells.

In another embodiment, a tissue scaffold may include a base layer comprising a pattern of pillars, a capping layer comprising a pattern of pillars, wherein the base pillars may be arranged into regions of microstructured pattern and the regions are arranged in a pattern, wherein the long-range energy gradients on the surfaces of the tissue scaffold may be biomimetic to extracellular matrix.

In another embodiment, a tissue scaffold may include a base layer comprising a pattern of pillars, a capping layer comprising caps of hydrophilic and hydrophobic polymers, the capping layer comprising zones of hydrophilic and hydrophobic caps which may be arranged in a pattern and the zones arranged in a long-range pattern, when the tissue scaffold is placed in a wet environment the surface may form a non-biologic cell adhesive layer disposed on the capping layer, and a component capable of generating an extracellular matrix that is associated with the long-range pattern of the microstructure zones. The scaffolds comprising a component for generating an extracellular matrix may further comprise living cells attached to the tissue scaffold.

Accordingly, in another embodiment, an artificial tissue may comprise living cells attached to a tissue scaffold having a base layer comprising a pattern of pillars, a capping layer comprising a pattern of hydrophilic/hydrophobic capped pillars, and a cultured extracellular matrix component.

In another embodiment, an artificial tissue may include living cells attached to a tissue scaffold comprising a base layer having a pattern of pillars, a capping layer comprising a pattern of hierarchically microstructured pillars, and wherein when placed in a wet environment, the artificial tissue may induce a non-biologic cell adhesive effect that may be at least in part generated by a Wenzel-Cassie interface with a target tissue surface.

In another embodiment, a method for making a tissue scaffold is provided wherein the method may include generating a pattern of pillars on a base layer by photolithography to form a patterned base layer and depositing a capping layer onto the patterned base layer to form a pattern of pillars capped individually with high surface energy and low surface energy polymer.

Methods of photolithography known in the art may be used to form the patterned base layer. For example, the method may comprise depositing a photoresist onto the base layer, thereby generating a photoresist layer, placing a mask on top of the photoresist layer and exposing the photoresist layer to ultraviolet radiation, thereby generating a patterned base layer. A patterned base layer made by photolithography may serve as a mold to mass produce patterned base layers.

In one embodiment, spin coating may be used to apply a photoresist layer onto the base layer. Spin coating is a process wherein the base layer may be, for example, mounted to a chuck under vacuum and may be rotated to spin the base layer about its axis of symmetry and a liquid or semi-liquid substance, e.g., a photoresist, may be dripped onto the base layer, with the centrifugal force generated by the spin causing the liquid or semi-liquid substance to spread substantially evenly across the surface of the base layer. Variations of this process, for example coating and then spinning, or spinning and then dripping, may also be used.

As used herein, the term “photoresist” may be understood to refer to any substance that may be sensitive to ultraviolet radiation, e.g., wavelengths of light in the ultraviolet or shorter spectrum (<400 um). It will be understood that a photoresist may be positive or negative.

In some embodiments, a base layer comprising a photoresist may be patterned by providing a mask comprising the desired shape and/or pattern, i.e., a dot pattern. The mask may be a solid mask such as a photolithographic mask. The mask may be provided and placed on top of the photoresist layer. Subsequently, a portion of the photoresist layer (i.e., the portion of the photoresist not covered by the mask) may be exposed to ultraviolet radiation.

In some embodiments, the mask placed on top of the photoresist layer may be typically fabricated by standard photolithographic procedure, e.g., by means of electron beam lithography. Other methods for creating such masks may be understood to include focused energy for ablation (micromachining) including lasers, electron beams and focused ion beams. Similarly, chemical etchants may be used to erode materials through the photoresist when using an alternative mask material.

Examples of chemical etchants include, but are not limited to, hydrofluoric acid and hydrochloric acid. Photolithographic masks are also commercially available.

In some embodiments, as disclosed herein, the microstructures on the patterned scaffold substrates may include features having one or more dimensions of less than 1 micrometer. The polymers comprising these microstructures can be deposited via soft lithography, for example. In some embodiments, the polymer can be printed on the transitional polymer with a polydimethylsiloxane stamp. Optionally, the process includes printing multiple polymer structures with different surface energies with successive, stacked printings. For example, where each polymer may be a protein, different proteins are may be printed in different printings.

In some embodiments, the polymer may be deposited via self-assembly on a transitional polymer. Exemplary self-assembly processes include, but are not limited to, assembly of collagen into fibrils, and assembly of actin into filaments, and assembly of DNA into double strands, for example.

In another aspect of some embodiments, the polymer may be deposited via vaporization of the polymer and/or deposition of the polymer through a mask onto the transitional polymer. For example, the polymer may be deposited via patterned photo-cross-linking on the transitional polymer and patterned light photo-cross-links the polymer in the selected pattern for some embodiments. The method may optionally include the step of dissolving non-cross-linked polymer outside the selected pattern in certain embodiments. The patterned light may change the reactivity of the polymer via release of a photolabile group or via a secondary photosensitive compound in the selected pattern.

In some embodiments, the method of capping a microstructure can include a step of allowing a polymer to bind together via a force selected from hydrophilic, hydrophobic, ionic, covalent, Van der Waals, hydrogen bonding, and/or via physical entanglement, and combinations thereof. The polymer structure may be released, in some embodiments, by applying a solvent to the transitional polymer to dissolve the transitional polymer or to change the surface energy of the transitional polymer, wherein the polymer structure may be released into the solvent as a freestanding structure. For example, the polymer may be released by applying a positive charge bias to the transitional polymer, by allowing the transitional polymer to undergo hydrolysis, or by subjecting the transitional polymer to enzymatic action. The polymer may be constructed in a pattern such as on an array of pillars. In some embodiments, a plurality of structures may be produced, e.g., the method includes a step of stacking a plurality polymer structure to produce a multi-layer, hierarchical microstructured tissue scaffold.

In some embodiments, the microstructure may include at least one dimension on the scale of 10 nanometers. In certain embodiments, capillary force lithography or nano-imprinting techniques may be used to fabricate highly uniform nanopatterned substrates on a large area. This robust technique can be used on a number of polymers with various chemical, physical, and electrical properties. This technique is scalable, and scaffold size (surface area) can vary from extremely small for smaller or individual cell cultures to large constructs of macroscopic tissue, depending on the embodiment employed.

In some embodiments, the nanostructures may be used to alter the surface energy of a microstructure surface residing on the soft tissue scaffold. In certain embodiments, the nanostructures can be ridges or pillars with a depth of between 10 nm-10 μm, or between about 50 nm-500 nm, or at least about 10 nm, or at least about 20 nm, or at least about 30 nm, or at least about 40 nm, or at least about 50 nm, or at least about 75 nm, or at least about 100 nm, or at least about 150 nm, or at least about 200 nm, or at least about 250 nm or at least about 500 nm, or at least about 1000 nm, or at least about 2000 nm, or at least about 3000 nm, or at least about 4000 nm, or at least about 5000 nm, or at least about 6 μm, or at least about 7 μm, or at least about 8 μm, or at least about 9 μm or at least about 10 μm.

In some embodiments, the depth may be between about 300 nm, but less than 1000 nm. In some embodiments, the depth of the groove may be between 5 nm-1000 nm (1 μm), for example at least about 5 nm, at least about 10 nm, at least about 20 nm, at least about 30 nm, at least about 40 nm, at least about 50 nm, at least about 60 nm, at least about 70 nm, or at least about 80 nm, or at least about 90 nm, at least about 100 nm, at least about 200 nm, at least about 300 nm, at least about 400 nm, or at least about 500 nm, or at least about 600 nm, or at least about 700 nm, or at least about 800 nm, or at least about 900 nm, or at least about 1000 nm (1 μm), but not more than 1000 nm (1 μm) in depth.

In some embodiments, the depth of the groove may be between about 200-800 nm, or between about 100-200 nm, or between about 200-400 nm, or between about 400-600 nm, or between about 600-800 nm, or between about 800-1000 nm (1 μm).

In some embodiments, the depth of the groove may be 200 nm. In some embodiments, the depth of the groove may be between 20-100 nm, or between about 20-50 nm, or between about 40-60 nm, or between about 50-75 nm, or between about 75-100 nm.

In some embodiments, the width of the groove may be between 50 nm-10 μm, or between about 200 nm-1000 nm, or at least about 50 nm, or at least about 75 nm, or at least about 100 nm, or at least about 150 nm, or at least about 200 nm, or at least about 250 nm or at least about 500 nm, or at least about 1000 nm, or at least about 2000 nm, or at least about 3000 nm, or at least about 4000 nm, or at least about 5000 nm, or at least about 6 μm, or at least about 7 μm, or at least about 8 μm, or at least about 9 μm or at least about 10 μm.

In some embodiments, the depth may be between about 300 nm, but less than 1000 nm. In some embodiments, a range for the groove width for maturing cardiomyocytes may be between about 200 nm-1000 nm.

In some embodiments, the width of the groove may be between 10-100 nm, for example at least about 10 nm, or at least about 20 nm, or at least about 30 nm, or at least about 40 nm, or at least about 50 nm, or at least about 60 nm, or at least about 70 nm, or at least about 80 nm, or at least about 90 nm, or at least about 100 nm or more than 100 nm in width.

In some embodiments, the width of the groove may be between 5 nm-1 μm, for example at least about 5 nm, at least about 10 nm, at least about 20 nm, at least about 30 nm, at least about 40 nm, at least about 50 nm, at least about 60 nm, at least about 70 nm, or at least about 80 nm, or at least about 90 nm, at least about 100 nm, at least about 200 nm, at least about 300 nm, at least about 400 nm, or at least about 500 nm, or at least about 600 nm, or at least about 700 nm, or at least about 800 nm, or at least about 900 nm, or at least about 1000 nm (1 μm), but not more than 1000 nm (1 μm) in width.

In some embodiments, the width of the groove may be between about 200-800 nm, or between about 100-200 nm, or between about 200-400 nm, or between about 400-600 nm, or between about 600-800 nm, or between about 800-1000 nm (1 μm).

In some embodiments, the width of the ridge may be between 50 nm-10 μm, or between about 200 nm-1000 nm, or at least about 50 nm, or at least about 75 nm, or at least about 100 nm, or at least about 150 nm, or at least about 200 nm, or at least about 250 nm or at least about 500 nm, or at least about 1000 nm, or at least about 2000 nm, or at least about 3000 nm, or at least about 4000 nm, or at least about 5000 nm, or at least about 6 μm, or at least about 7 am, or at least about 8 μm, or at least about 9 μm or at least about 10 μm. In some embodiments, the depth may be between about 300 nm, but may be less than 1000 nm.

In some embodiments, the ridges between the grooves have a width of about 50 nm-1 μm (1000 nm), for example, at least about 50 nm, or at least about 60 nm, or at least about 70 nm, or at least about 80 nm, or at least about 90 nm, or at least about 100 nm, or at least about 125 nm, or at least about 150 nm, or at least about 175 nm, or at least about 200 nm, or at least about 250 nm, or at least about 300 nm, or at least about 400 nm, or at least about 500 nm, or at least about 600 nm, or at least about 700 nm, or at least about 800 nm, or at least about 900 nm, or at least about 50 nm or any integer between 50 nm and 1 μm (1000 nm), but not more than 1000 nm.

In some embodiments, the width of the ridges may be 150 nm. In some embodiments, the width of the ridge may be between 5 nm-1 μm, for example at least about 5 nm, at least about 10 nm, at least about 20 nm, at least about 30 nm, at least about 40 nm, at least about 50 nm, at least about 60 nm, at least about 70 nm, or at least about 80 nm, or at least about 90 nm, at least about 100 nm, at least about 200 nm, at least about 300 nm, at least about 400 nm, or at least about 500 nm, or at least about 600 nm, or at least about 700 nm, or at least about 800 nm, or at least about 900 nm, or at least about 1000 nm (1 μm), but not more than 1000 nm (1 μm) in width. In some embodiments, the width of the groove may be between about 200-800 nm, or between about 100-200 nm, or between about 200-400 nm, or between about 400-600 nm, or between about 600-800 nm, or between about 800-1000 nm (1 μm).

Any suitable material, e.g., a material that has a flat surface, e.g., a metal (gold, silver, platinum, tantalum, or aluminum), a ceramic (alumina, titanium oxide, silica, or silicon nitride), may be used for making the mask.

In some embodiments, a combination of positive and negative photoresists can be used. For example, a positive photoresist may be deposited on a base layer in a particular pattern and subsequently a negative photoresist in a complementary pattern may be applied. This results in a patterned tissue scaffold that comprises a pattern that comprises regions that are of a particular surface energy next to regions that are of a different surface energy.

Once the photoresist layer is exposed to ultraviolet radiation and a patterned base layer is formed, the mask may be removed and a capping substance may be deposited to the patterned base layer to form an inter-microstructure patterned capping layer. The capping substance binds directly onto the base layer and does not depend on the introduction of reactive side chain-containing species into the base layer.

In one embodiment, a thin capping layer, e.g., a silicone polymer, may be deposited onto the patterned base layer as a continuous layer on each microstructure of the patterned base layer.

In one embodiment, capping molecules, e.g., hydrophobic molecules, may be bonded together on at least a portion of the microstructures of the patterned base layer to form a continuous layer. In another embodiment, a capping layer, e.g., hydrophobic molecules, may be deposited onto the microstructures of the patterned base layer as a non-continuous layer, i.e., a pattern of individual molecules covering the surface.

The capping substance may be deposited onto the microstructures of the patterned base layer under conditions suitable to form a capping layer on the patterned base layer. This may be achieved using any suitable technique known to one of ordinary skill in the art and includes, for example, an xy-translation device which spot-prints the capping substance individually on the microstructures. In some embodiments, the step of forming a patterned capping layer may include subjecting a continuous capping layer to pyrolysis, microwaving, complete hydrolysis or partial hydrolysis.

In an embodiment, the patterned capping layer may be about 0.1 to about 100 um, 0.1 to about 70 um, about 0.1 to about 50 um, about 0.1 to about 30 um, 0.1 to about 20 um, about 0.1 to about 10 um, may be about 0.1 to about 10 um, 0.1 to about 7 um, about 0.1 to about 5 um, about 0.1 to about 3 um, 0.1 to about 2 um, about 0.1 to about 1 um, about 0.5 to about 2 um, about 1 to about 2 um, about 1 to about 1.5 um, about 1.5 to about 2 um, or about 0.1 um, 0.5 um, 1 um, 1.1 um, 1.2 um, 1.3 um, 1.4 um, 1.5 um, 1.6 um, 1.7 um, 1.8 um, 1.9 um, 2 um, 2.1 um, 2.2 um, 2.3 um, 2.4 um, 2.5 um, 2.6 um, 2.7 um, 2.8 um, 2.9 um, 3.0 um, 3.1 um, 3.2 um, 3.3 um, 3.4 um, 3.5 um, 3.6 um, 3.7 um, 3.8 um, 3.9 um, 4.0 um, 4.1 um, 4.2 um, 4.3 um, 4.4 um, 4.5 um, 4.6 um, 4.7 um, 4.8 um, 4.9 um, 5.0 um, 5.1 um, 5.2 um, 5.3 um, 5.4 um, 5.5 um, 5.6 um, 5.7 um, 5.8 um, 5.9 um, 6.0 um, 6.1 um, 6.2 um, 6.3 um, 6.4 um, 6.5 um, 6.6 um, 6.7 um, 6.8 um, 6.9 um, 7.0 um, 7.1 um, 7.2 um, 7.3 um, 7.4 um, 7.5 um, 7.6 um, 7.7 um, 7.8 um, 7.9 um, 8.0 um, 8.1 um, 8.2 um, 8.3 um, 8.4 um, 8.5 um, 8.6 um, 8.7 um, 8.8 um, 8.9 um, 9.0 um, 9.1 um, 9.2 um, 9.3 um, 9.4 um, 9.5 um, 3.6 um, 9.7 um, 9.8 um, 9.9 um, or about 10.0 um in thickness.

In another embodiment, the patterned chemical layer may be 2 um or less in thickness. In an embodiment, the patterned chemical layer may be about 1 to about 1.5 um in thickness. In an embodiment, the patterned chemical layer may be about 10 to about 70 nm in thickness. In another embodiment, multiple layers of a semi-rigid or soft polymer are coated on the base layer.

In some embodiments, the patterned layer can be about 1 to about 50, about 1 to about 45, about 1 to about 40, about 1 to about 35, about 1 to about 30, about 1 to about 25, about 1 to about 20, about 1 to about 15, about 1 to about 10, about 1 to about 5, about 2 to about 50, about 2 to about 45, about 2 to about 40, about 2 to about 35, about 2 to about 30, about 2 to about 25, about 2 to about 20, about 2 to about 15, about 2 to about 10, about 2 to about 5, about 5 to about 50, about 5 to about 45, about 5 to about 40, about 5 to about 35, about 5 to about 30, about 5 to about 25, about 5 to about 20, about 5 to about 15, about 5 to about 10, about 10 to about 50, about 10 to about 45, about 10 to about 40, about 10 to about 35, about 10 to about 30, about 10 to about 25, about 10 to about 20, about 10 to about 15, about 20 to about 50, about 25 to about 50, about 30 to about 50, about 35 to about 50, about 40 to about 50, or about 45 to about 50 nm monolayers thick. Ranges and values intermediate to the above recited ranges and values are also contemplated to be part of the disclosure. For example, ranges for 1-5 and 7-25 nm monolayer thicknesses are intended to be encompassed by the present disclosure. Other examples are also contemplated by this disclosure based on the ranges and values recited above.

In some embodiments, a tissue scaffold can further comprise a cytokine layer disposed about the capping layer. In certain embodiments, a cell signaling chemical compound may be deposited onto the patterned capping layer to form a patterned cell signaling layer.

In some embodiments, an avoidance layer may be deposited on all or a portion of the patterned capping layer. In certain embodiments, an avoidance layer may be deposited in a pattern that may be complementary to a cell cytokine layer. In certain embodiments, an avoidance layer may be a layer that inhibits the adhesion of cells, bacteria, or viruses.

Compounds that may be considered suitable for use as a microbe-avoidance layer include, but are not limited to, compounds with terminal pegylated groups and those comprising an alkyl terminal group.

In certain embodiments, the tissue scaffolds may further comprise living cells. Accordingly, in certain embodiments, a method for making an artificial tissue may include living cells attached to a tissue scaffold, the method comprising contacting a tissue scaffold with cells and culturing under conditions suitable for cell growth and/or differentiation.

The type of cells suitable for these embodiments includes, but is not limited to, and fibroblasts, endothelial cells, keratinocytes, osteoblasts, chondroblasts and chondrocytes, hepatocytes, macrophages, cardiac muscle cells, smooth muscle cells, skeletal muscle cells, tendon cells, ligament cells, neural cells, epithelial cells, and stem cells. Stem cells may include embryonic stem cells, adult stem cells, and induced pluripotent stem cells. In one embodiment, the cells are mesenchymal stem cells.

In another embodiment, the cells are human cells. Culture conditions for cell growth and/or differentiation are known to those of skill in the art.

In yet another embodiment, a method for making a tissue scaffold may include the method comprising generating a pattern of microstructures on a base layer by photolithography to form a substrate having patterned base layer, depositing a capping layer onto the patterned base layer to form a substrate having a patterned capping layer, contacting this synthetic tissue scaffold with cells and culturing under conditions suitable for the production of extracellular matrix components, and removing the cells from the substrate to provide a tissue scaffold comprising an extracellular matrix component generated by the long-range structure of the microstructures.

In some embodiments, the capping layer may be the result of nanotopography and of any conformation and geometry of parallel grooves and ridges that allows for anisotropic and polarized cell arrangement in the direction of the nanotextures. In particular, the polarization of the capping microstructure may correspond to the long-range structures expected as a result of coded zones.

In some embodiments, the top portion, or the surface, of the ridge may be substantially planar, and in some embodiments, it may be convex, and in some embodiments, it may be concave. In some embodiments, the ridge may be pointed or angular.

In some embodiments, the hollow portion, or bottom of the microstructure, may be substantially planar, and in some embodiments, it may be concave, and, in some embodiments, it may be convex. Any combination of planar, convex or concave surfaces of the groves and ridges can occur and is contemplated, although it may be generally preferred that the nanotextured area has a repeating unit of the same geometry.

In some embodiments, the surface of the ridges and/or grooves all have the same geometry, e.g., they are all substantially planar. In alternative embodiments, the ridges and/or grooves have a variety of a combination of convex, concave, or substantially planar surfaces. In some embodiments, the ridges and grooves are convex and concave respectively, to provide a corrugated cross-sectional appearance.

In some embodiments, after production of extracellular matrix components, the cells may be removed from the synthetic tissue scaffold comprising a substrate, i.e., the substrate may be decellularized.

Methods for decellularizing are known in the art and include, for example, methods to loosen cell attachments from the extracellular matrix followed by lysis of cell membranes and solubilization of intracellular components under conditions that maintain the integrity and activity of the matrix. For example, decellularization may be accomplished by treatment to remove calcium by chelation to loosen cell attachments, followed by incubation with non-ionic detergent in a hypotonic buffer at alkaline pH to lyse cell membranes and solubilize intracellular components.

The scaffolds comprising an extracellular matrix component may further comprise living cells attached to the matrix component. Accordingly, in another embodiment, a method for making an artificial tissue comprising living cells attached to a tissue scaffold may include the method comprising contacting a tissue scaffold with cells and culturing under conditions suitable for cell growth and/or differentiation.

In some embodiments, cells may be attached to the tissue scaffold substrate by placing the scaffold in culture with a cell suspension and allowing the cells to settle and adhere to the surface. Cells may respond to the patterning of the scaffold surface in terms of adherence and in terms of assembling ECM proteins in the pattern on the scaffold surface. Cells also respond to the patterning in terms of maturation, growth and function. The cells on the scaffold may be cultured in an incubator under physiologic conditions (e.g., at 37° C.) until the cells form a two-dimensional tissue, the orientation of which may be determined by the pattern provided on the tissue scaffold.

Appropriate cell culture methods may be used to establish the tissue on the tissue scaffold, which are generally known in the art. The seeding density of the cells will vary depending on the cell size and cell type but can easily be determined by methods known in the art. In one embodiment, cells are seeded at a density of between about 1×103 to about 6×105 cells/cm², or at a density of about 1×103 cells/cm², about 2×103 cells/cm², about 3×103 cells/cm², about 4×103 cells/cm², about 5×103 cells/cm², about 6×103 cells/cm², about 7×103 cells/cm², about 8×103 cells/cm², about 9×103 cells/cm², about 1×104 cells/cm², about 2×104 cells/cm², about 3×104 cells/cm², about 4×104 cells/cm², about 5×104 cells/cm², about 6×104 cells/cm², about 7×104 cells/cm², about 8×104 cells/cm², about 9×104 cells/cm², about 1×105 cells/cm², about 1.5×105 cells/cm², about 2×105 cells/cm², about 2.5×105 cells/cm², about 3×105 cells/cm², about 3.5×105 cells/cm², about 4×105 cells/cm², about 4.5×105 cells/cm², about 5×105 cells/cm², about 5.5×105 cells/cm², about 6×105 cells/cm², about 6.5×105 cells/cm², about 7×105 cells/cm², about 7.5×105 cells/cm², about 8×105 cells/cm², about 8.5×105 cells/cm², about 9×105 cells/cm², about 9.5×105 cells/cm², about 1×106 cells/cm², about 1.5×106 cells/cm², about 2×106 cells/cm², about 2.5×106 cells/cm², about 3×106 cells/cm², about 3.5×106 cells/cm², about 4×106 cells/cm², about 4.5×106 cells/cm², about 5×106 cells/cm², about 5.5×106 cells/cm², about 6×106 cells/cm², about 6.5×106 cells/cm², about 7×106 cells/cm², about 7.5×106 cells/cm², about 8×106 cells/cm², about 8.5×106 cells/cm², about 9×106 cells/cm², or about 9.5×106 cells/cm². Values and ranges intermediate to the above-recited values and ranges are also contemplated.

In one embodiment the patterned tissue scaffold may be contacted with a plurality of cells and cultured such that a living tissue, e.g., a tissue having at least in part, in vivo biological activity, may be produced. In one embodiment, a living tissue may be removed from the tissue scaffold.

In some embodiments, the CMS polymer substrate useful in the compositions for the methods described herein can be sterilized using conventional disinfection/sterilization techniques including glutaraldehyde tanning, formaldehyde tanning at acidic pH, propylene oxide treatment, ethylene oxide treatment, gas plasma sterilization, gamma irradiation or electron beam treatment, and peracetic acid (PAA) disinfection.

Sterilization techniques which do not adversely affect the mechanical strength, structure, and biotropic properties of the polymer substrate may be preferred, though others may also be useful and sufficient. For instance, strong gamma irradiation can cause loss of strength of the sheets of polymer substrates. Sterilization techniques include exposing the polymer substrate to peracetic acid, 1-4 Mrads gamma irradiation (more preferably 1-2.5 Mrads of gamma irradiation) or gas plasma sterilization. In some instances, a polymer substrate can be subjected to two or more sterilization processes. After the polymer substrate is treated in an initial disinfection step, for example by treatment with peracetic acid, the polymer substrate can be wrapped in a plastic or foil wrap and sterilized again using electron beam or gamma irradiation sterilization techniques.

The patterned tissue scaffolds (and/or a living tissue prepared on a tissue scaffold and removed from the scaffold) may be used in a broad range of applications, including, but not limited to, devices for use in tissue repair and support such as sutures, surgical and orthopedic screws, and surgical and orthopedic plates, natural coatings or components for synthetic implants, cosmetic implants and supports, repair or structural support for organs or tissues, substance delivery, bioengineering platforms, platforms for testing the effect of substances upon cells, cell culture, wound healing, and numerous other uses.

In one embodiment, the living tissue may be removed from the scaffold prior to use. In another embodiment, the living tissue may be not removed from the scaffold prior to use.

The base layer of the patterned tissue scaffolds may be tissue prepared on the tissue scaffolds or a medical device, such as an orthopedic screw or plate that comprises cells of the same tissue in which the devices will be used. Non-limiting examples of medical devices suitable for use include, diagnostic implant devices, biosensors, stimulators, diabetic implants such as glucose monitoring devices, external fixation devices, external fixation implants, orthopedic trauma implants, implants for use in joint and spinal disorders/reconstruction such as plates, screws, rods, plugs, cages, scaffolds, artificial joints (e.g., hand, wrist, elbow, shoulder, spine, hip, knee, ankle), wires and the like, oncology related bone and soft tissue replacement devices, dental and oral/maxillofacial devices, cardiovascular implants such as stents, catheters, valves, rings, implantable defibrillators, and the like, contact lenses, ocular implants, keratoprosthesis, dermatologic implants, cosmetic implants, implantable medication delivery pumps; general surgery devices and implants such as but not limited to drainage catheters, shunts, tapes, meshes, ropes, cables, wires, sutures, skin staples, burn sheets, and vascular patches; and temporary/non-permanent implants.

In addition, because the methods described herein are applicable to soft polymeric base layers, flexible membranes comprising a patterned tissue scaffold may be used to support or connect tissue or structures that have experienced injury, surgery, or deterioration. For example, a patterned tissue scaffold comprising a soft polymer may be used as a graft to connect and/or bind tissue and provide a platform for tissue regeneration, internally or externally. In such instances, the soft polymer may be biodegradable or non-biodegradable.

Another use of the patterned soft tissue scaffolds may be as a barrier for the prevention of post-operative induced adhesion(s). For example, because adhesions are the result of disorganized ECM, the patterned tissue scaffolds may be used to organize ECM deposition and prevent the formation of adhesions.

Yet another embodiment of the patterned tissue scaffolds may be as templates for nerve growth. For example, the patterned tissue scaffolds may be used to culture neural cells in a pattern mimicking the in vivo environment such that suitable neural connections form rather than the unorganized array of neural cells that are produced without use of a patterned scaffold.

Accordingly, in one embodiment, methods of tissue repair and regeneration may include implanting the artificial tissues in a subject in need of such tissue repair or regeneration.

In some embodiments, patterned tissue scaffolds contacted or seeded with living cells may be combined with a drug such that the function of an implant or graft may be improved. For example, antibiotics, anti-inflammatories, local anesthetics, or combinations thereof, may be added to the cell-treated patterned tissue scaffold to affect the healing process in a positive way.

In one embodiment, the tissue scaffolds may be used to study functional differentiation of stem cells (e.g., pluripotent stem cells, multipotent stem cells, induced pluripotent stem cells, and progenitor cells of embryonic, fetal, neonatal, juvenile and adult origin). For example, the patterned tissue scaffolds may be contacted with undifferentiated cells, e.g., stem cells, and differentiation may be observed.

Patterned tissue scaffolds seeded with cells and cultured to form a tissue are also useful for measuring tissue activities or functions, investigating tissue developmental biology and disease pathology, as well as in drug discovery.

Accordingly, the present disclosure also provides methods for identifying a compound that modulates a tissue function. The methods may include providing a tissue scaffold comprising a tissue produced according to the methods of the disclosure, contacting the tissue with a test compound; and measuring the effect of the test compound on a tissue function in the presence and absence of the test compound, wherein a modulation of the tissue function in the presence of the test compound as compared to the tissue function in the absence of the test compound may indicate that the test compound modulates a tissue function, thereby identifying a compound that modulates a tissue function.

In another aspect, the present disclosure also provides methods for identifying a compound that may be useful for treating or preventing a disease. In certain embodiments, the methods include providing a tissue scaffold comprising a tissue produced according to any one of the methods of the disclosure, contacting a tissue with a test compound, and measuring the effect of the test compound on a tissue function in the presence and absence of the test compound, wherein a modulation of the tissue function in the presence of the test compound as compared to the tissue function in the absence of the test compound indicates that the test compound modulates a tissue function, thereby identifying a compound useful for treating or preventing a disease.

In some embodiments, the test compound or compounds used, may be any agents including chemical agents (such as toxins), small molecules, pharmaceuticals, peptides, proteins (such as antibodies, cytokines, enzymes, and the like), and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like, and combinations thereof.

In certain embodiments, the test compound may be added to a tissue by any suitable means. For example, the test compound may be added dropwise onto the surface of a tissue scaffold and allowed to diffuse into or otherwise enter the tissue, or it can be added to the nutrient medium and allowed to diffuse through the medium.

The following examples serve to further illustrate various embodiments as disclosed herein and should not be taken as limiting or exhaustive of the disclosure.

### Example 1. Surface Energy Gradients of a Tissue Scaffold Having Microstructure Pattern

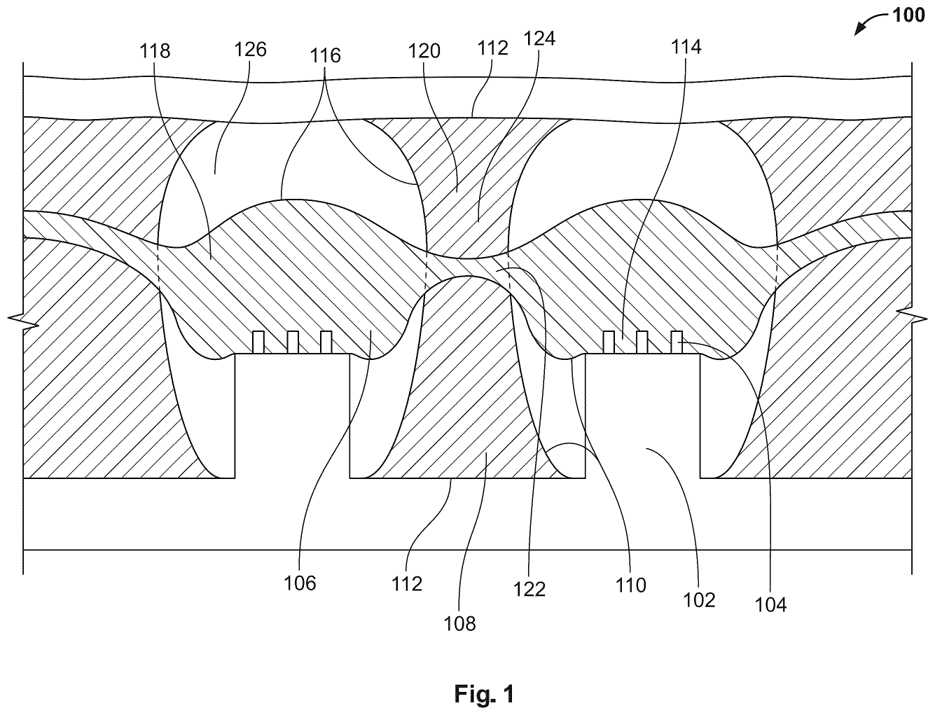

In one embodiment, the tissue scaffolds includes a microstructure surface and chemical modification of the microstructure surface to generate patterns of surface energy gradients. Surface energy of liquids may be reported as surface tension which may be energy per unit surface area. For a solid, the surface energy in a given plane projected area is proportional to the surface area of the solid in the plane projected area. The variation of energy per unit area as a function of a distance is a surface energy gradient. Referring to FIG. 1, a pillar-on-pillar microstructure surface 100 comprises first pillars 102 and second pillars 104 and in contact with a binary fluid comprised of a high surface tension liquid 106 and a low surface tension liquid 108. Liquids 106 and 108 may be immiscible in some embodiments. A surface energy gradient 110 may result from the high surface energy 112 and low surface energy 114, wherein continuity requires the surface energy to vary smoothly from area of high surface energy 112 to area of low surface energy 114, thus resulting in a surface energy gradient. Liquids 106, 108 map this energy gradient by developing interfacial surfaces 116. These interfacial surfaces result in the formation of capillary bridges 118, which bridges form both within the tissue scaffold and between the tissue scaffold and a tissue surface. Capillary bridge 122 may be within tissue scaffold capillary bridge. Capillary bridge 124 may be a capillary bridge from the tissue scaffold 118 to the tissue 120. Zone 126 comprises a mixture of liquids 106, 108. In zone 126 the energy gradients are not large enough to cause separation of the liquids 106, 108. Depending on environmental conditions, zone 126 may resolve into separation of liquids 106, 108. As a result the two capillary bridge types 122, 124 share interfacial surfaces 116. This structure of interfacial surfaces 116 may be self-stabilizing against thermal disruption and acts as guiding surfaces to direct cells into topological arrangements. Cells of different types may follow different paths along surfaces 116.

In some embodiments disclosed herein, the tissue scaffolds may guide cells into arrangements that act as precursor structures for long-range tissue formation. These guiding surfaces 116 may direct cells to generate functional tissue within the scaffold 100 and between the scaffold and surrounding tissue 120, thus providing a means for directing neovascularization from living tissue 120 to scaffold tissue. Conventional tissue scaffolds are often encapsulated in dense fibrous tissue, separating the tissue formed on the tissue scaffold from a nutrient and oxygen source.

### Example 2. Coded Zones of a Tissue Scaffold Having a Microstructure Pattern

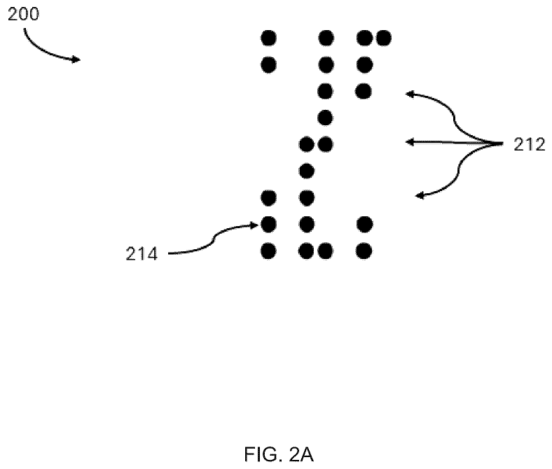

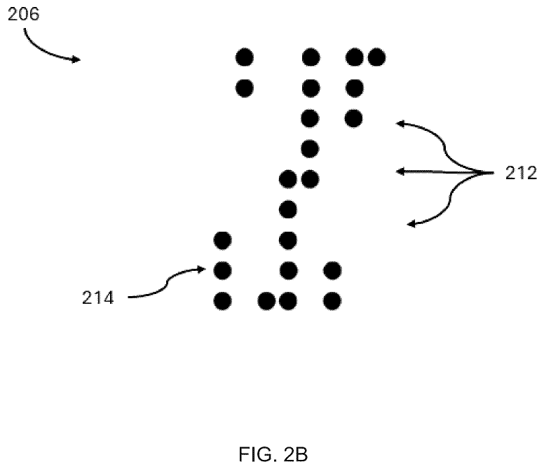

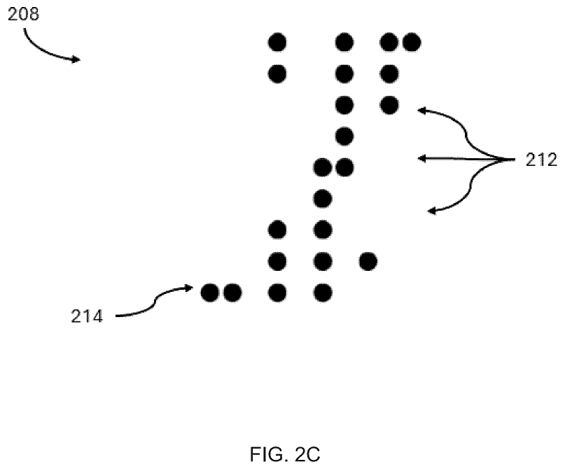

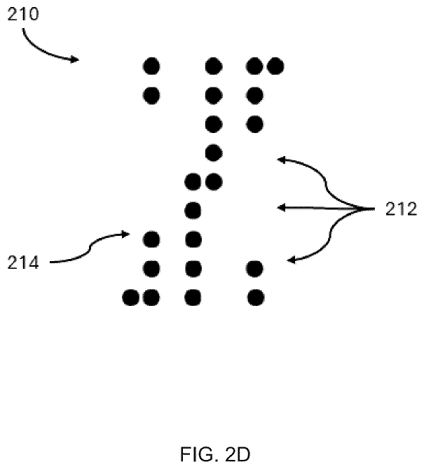

Referring to FIG. 2a, an illustration of a code is depicted. The term “code” may be understood to refer to a microstructure pattern comprising a juxtaposition of surface energy gradients in a defined surface area. In a simplistic embodiment of a microstructure surface 200, the surface energy gradients of the microstructure pattern may be defined by microstructure surfaces of different surface energy. For example, a low surface energy (hydrophobic) microstructure may be juxtaposed with a high surface energy (hydrophilic) microstructure. Labeling these microstructures as 0 for a hydrophobic microstructures and as 1 for a hydrophilic microstructures, an array of 0's and 1's may be understood to describe a code. The array can be two dimensional, or higher dimensional when two-dimensional microstructured surfaces are stacked.

Considering a two-dimensional code, the zone defining the code may be comprised of lines of 0's and 1's. Considering a first line as an object, then the adjacent line can be considered a permutation of the first line. Consequently, a code may be specified as a first line of 0's and 1's and a permutation group acting on a specified first line. Selecting different first lines will generate different zones of code for the same group operation. Thus, the zones specified by a code may represent the elements of a mathematical group.

For a given first line, a set of coded zones can be generated by a first mathematical group operation. These first zones can be treated as elements of another second group and larger second zones can be constructed by applying the second group operation to the first zones. There are many variations on this theme. When the same group is applied repeatedly to first, second, etc. zones a self-similar structure may be obtained. These self-similar structures may be macroscopic, and mimic many natural tissue architectures.

Mathematical groups of certain embodiments may comprise Zn: the cyclic group of order n (the notation Cn is also used; it may be isomorphic to the additive group), the dihedral group of order 2n (often the notation Dₙ or D₂ₙ is used), K₄: the Klein four-group of order 4, Sn: the symmetric group of degree n, containing the n! permutations of n elements, A.: the alternating group of degree n, containing the even permutations of n elements, of order 1 for n=0, 1, and order n!/2 otherwise, Q₄ₙ: the dicyclic group of order 4n, and Q8: the quaternion group of order 8.

Referring to FIG. 2a-d, an example of a set of microstructure surface zones 200, 206, 208, 210 including a zone of microfeatures that may embody a group operation which acts as a long-range organizing language to cells. When these zones are juxtaposed, the language encodes different cellular arrangements.

For example, in FIG. 2a, the microstructure surface zone 200 illustrates an embodiment that may include lines of microstructure 212. The microstructure surface zone 200 may be comprised of 9 lines of microstructure 212. Each line of microstructure 212 may be comprised of microstructure elements 214, with each line 212 comprising up to 8 μmicrostructure elements 214. For example, microstructure element 214 may be of the hydrophobic kind, or of the hydrophilic kind. One simple embodiment comprises microstructure elements 214 which are circular pillars capped with in one case a hydrophilic material and in another case a hydrophobic material. In constructing the second order microstructure, two zones of a microstructure surface, 200, 210 can be considered hydrophilic and hydrophobic, respectively. Then the surface zones 200, 210 can be juxtaposed to make a larger zone by arranging the surfaces according to the code in surface zone 206. The construction involves replacing the hydrophilic and hydrophobic microstructures of surface zone 206 with hydrophilic and hydrophobic zones of surfaces 200, 210. Accordingly, the 9×8 zone of microstructures of 206 becomes the 64×81 zone of microstructures of a larger zone. It should be appreciated that different combinations of these operations comprise different cellular instructions residing within a group defined cell signaling language.

### Example 3. Two-Dimensional Fourier Transforms of Zones of a Patterned Tissue Scaffold

A two-dimensional Fourier transform of an image plots spatial frequency distribution of black pixels in a black and white image. Fourier transforms provide a visual representation of the periodic structure cells sense as they proliferate on a patterned scaffold comprising zones as illustrated in FIG. 2.

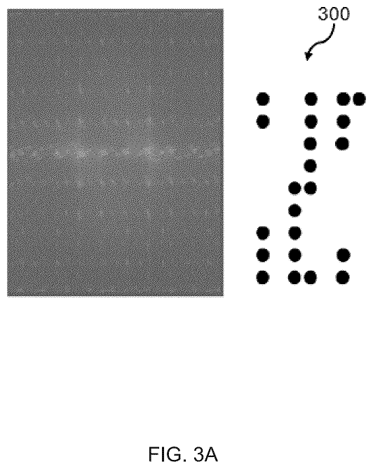

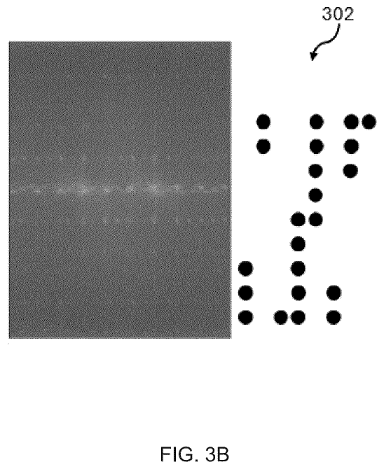

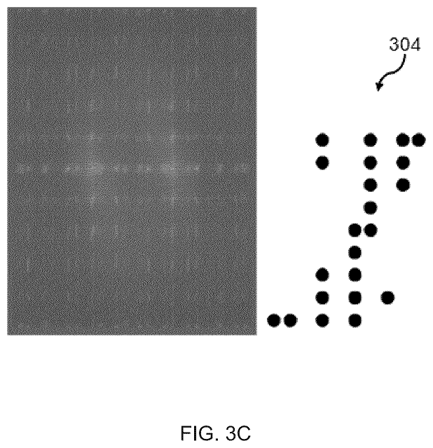

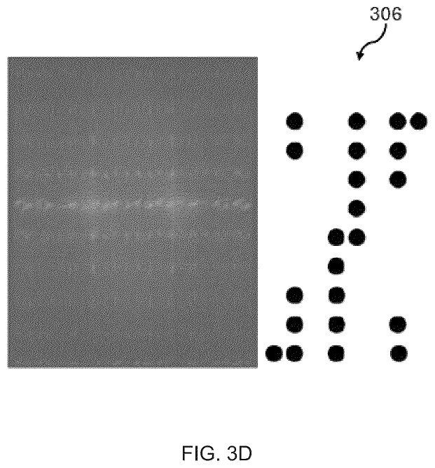

Referring to FIG. 3a-d, a language comprising Fourier transformed words may be useful in the present tissue scaffolds. The FFT images of FIG. 3 display the absolute value (or complex magnitude) of the spatial frequencies found in the image. The amplitude of the frequency coefficient may be displayed as intensity and the wavelength or frequency as its radial distance from the center of the image with low frequency pixels in the center and high frequency at the edge of the image. The orientation of the frequencies can also be seen in the 2D FFT perpendicular or orthogonal to the image feature in the real image, so for instance a horizontal line will be observed in the 2D FFT as a vertical feature.

Note, the microstructure surface zones 300, 302, 304, and 306 are similar macroscopically and different in fine detail. The macroscopic similarity between microstructure surface zones 300, 302, 304 and 306 may enable cells to proliferate over large spatial dimensions in a directed way. The fine detail differences between zones 300, 302, 304 and 306 may instruct cells to make different topological structures which act as precursors to mature functional tissue.

### Example 4. Two-Dimensional Fourier Transforms of Cells Grown on a Patterned Tissue Scaffold

To generate a second order tissue, Fast Fourier Transform was performed on a cell culture of an endothelial cell colonized a ATC GaYaTri block, stained blue.

The FFT images display the absolute value (or complex magnitude) of the spatial frequencies found in the image. The amplitude of the frequency coefficient may be displayed as intensity and the wavelength or frequency as its radial distance from the center of the image with low frequency pixels in the center and high frequency at the edge of the image. The orientation of the frequencies can also be seen in the 2D FFT perpendicular or orthogonal to the image feature in the real image, so for instance a horizontal line will be observed in the 2D FFT as a vertical feature.

### Example 5. Three-Dimensional Tissue Scaffold

Tissue scaffolds, in some embodiments, are biocompatible (bioresorbable or nonabsorbable) scaffolds of layered films, at least some of which are porous (macroporous or microporous) and that can provide controlled morphological guidance and material- and microstructure-based surface energy gradients.

The films used in certain embodiments of patterned tissue scaffolds may have a long-range microstructure that provides organizing energy gradients at the microstructure level that facilitate cellular invasion, proliferation, and differentiation that can ultimately result in regeneration of wholly or partially functional tissue. The films of the tissue scaffold may have patterns of surface energy gradients and patterns of microstructure that permits tissue ingrowth, tissue repair, tissue regeneration, and cell-based research for therapeutic agent discovery. The scaffold may provide layered films that have openings that provide interconnection between living cells organized on patterned films to control growth in a predictable manner.

The features of certain embodiments of patterned scaffolds may be controlled to suit a desired application by choosing the appropriate conditions to form a layered film structure with openings in select areas of each film. These patterned scaffolds may be arranged in layers having distinct advantages over the prior art where the scaffolds are isotropic or random structures.

The tissue scaffolds described herein may include cell openings (e.g., cell openings defining pores in one or more films) that vary in size and shape. Whether of a regular or irregular shape, the diameter of the cell opening can be between about 1 to about 10,000 microns. For example, cell openings can be from about 5 microns to 9,5000 microns; from about 10 to 10,000 microns; from about 25 to about 7,500 microns; from about 50 to 5,000 microns; from about 100 to about 2,500 microns; from about 100 to about 5,000 microns; from about 250 to about 2,500 microns; from about 250 to about 1,000 microns; from about 500 to about 1,000 microns; from about 750 to about 1,000 microns; or ranges therebetween.

The cellular openings can provide pathways for cellular ingrowth and nutrient diffusion in some embodiments. Porosities can be controlled and can range from about 10% to 95% porous. Because the cell openings and/or channels can have diameters in the range of microns, useful films and scaffolds can be described as microporous in some embodiments. They can also be non-porous and resorbable on certain embodiments.

The features of some embodiments of tissue scaffolds can be controlled to suit desired applications by selecting features to obtain surface energy gradients along three axes for preferential cell culturing; channels that run through the scaffold for enhanced cell invasion, vascularization, and nutrient diffusion; micro-patterning of films on the surface for improved cellular organization; tailor-ability of pore size and shape; anisotropic mechanical properties; composite layered structure with a polymer composition gradient to modify the cellular response to different materials; blends of different polymer compositions to create structures that have portions that will degrade or resorb at different rates; films blended or coated with bioactive agents, including but not limited to biological factors, growth factors, and the like; ability to make three dimensional structures with controlled microstructures; and assembly with other medical devices or agents to provide a composite structure.

In some embodiments, a biocompatible scaffold may include a substantially controllable pore structure designed to facilitate cellular linkage between layers of patterned tissue scaffold in a way reflecting the long-range patterns residing on each patterned tissue scaffold layer comprising a three-dimensional tissue scaffold assembly. Characteristics selected from the group comprising composition, stiffness, pore architecture, and biosorption rate can be controlled.

The scaffold can be made from absorbable or nonabsorbable polymers. A blend of polymers can be applied to form a compositional and surface energy gradient from one layer to the next. In applications where one composition may be sufficient, the scaffold provides a biocompatible scaffold that may have structural variations across one or more layers that may be capable of directing cellular infiltration and association on a long-range scale. The structural variations can result in a variation in degradation across the scaffold.

In some embodiments, the patterned three-dimensional tissue scaffold includes interconnecting pores and channels to facilitate the transport of nutrients and/or invasion of cells into layers of patterned tissue scaffold. Some channels may be created to facilitate delivery of agents, compounds, or cells into the scaffold using delivery means. Positive or negative pressure methods can be employed to deliver the agents, compounds, or cells.

In one aspect, a method for the repair or regeneration of tissue may include contacting a first tissue with a scaffold pore gradient at a location on the scaffold that has appropriate characteristics to permit growth of the tissue. The concept of controlled transition in physical and chemical properties, and/or microstructural features in the scaffold can facilitate the growth or regeneration of tissue.

The scaffolds are particularly useful for the generation of tissue junctions between two or more layers of tissue. For a multi-cellular system, one type of cell can be present in one area of the scaffold and a second type of cell can be present in a separate area of the scaffold. Delivery channels can be utilized to position agents, compounds or cells in certain regions of the scaffold. Channels can also be used to generate controlled flow of a medium using positive or negative pressure means. An external source can be used to generate flow through the channels.

A gradient of absorbable polymers of different layers forming a compositional gradient from one polymeric material to a second polymeric material can be created in some embodiments. In situations where one composition may be sufficient for the application, the scaffold may provide a biocompatible film scaffold that may have microstructural variations in the structure across one or more dimensions that may mimic the anatomical features of tissue. The cross-sectional area of the implant can vary in this instance. When the scaffold degrades by surface erosion or through bulk degradation, the regions with an increased cross-sectional area may degrade at a slower rate.

Films can be layered and bonded together in some embodiments. The films can be attached using ionic or covalent bonds. Photo-initiated bonds can be created using suitable materials such as benzophenone. Biocompatible adhesives can be used in certain embodiments.

Alternatively, heat and pressure can be used in certain embodiments.

In one case the material used to produce the patterned scaffold may comprise a sheet. The sheet may be substantially planar. The material may be at least partially of a layered construction in some embodiments. In one embodiment the material may comprise a first layer and a second layer, the first layer having a higher absorption rate than the second layer. The first layer may be located adjacent to the second layer. The second layer may be configured to be located closer to a tissue structure than the first layer.

In one embodiment the material may be at least partially porous to promote tissue in-growth. The first layer may have a higher pore density than the second layer. The first layer may have a smaller pore size than the second layer. In one case at least some of the pores form at least a partial gradient with varying density.

In another embodiment the material may be at least partially porous to promote tissue in-growth. The layers may have a higher pore density in select regions. The central region may have a higher pore density than the outer region. In one case at least some of the pores form at least a partial gradient from one region to the next.

The material may comprise an anti-adhesion filler filling at least some of the pores. The material may comprise an anti-adhesion coating along at least part of the surface of the material. Alternatively, a material used to promote tissue attachment and bonding may be utilized with the scaffold.

A “biocompatible substrate” as used herein may be understood to include a material that may be suitable for implantation into a subject. A biocompatible substrate reduces or may not cause toxic or injurious effects once implanted in the subject. In one embodiment, the biocompatible substrate may be a polymer with a surface that can be shaped into the desired structure that requires repair or replacement. The polymer can also be shaped into a part of a structure that requires repair or replacement. The biocompatible substrate provides the supportive framework that allows cells to attach to it and grow on it. Cultured populations of cells can then be grown on the biocompatible substrate, which includes the microstructured substrate that provides the appropriate interstitial distances required for cell-cell interaction.

In some embodiments, an CMS substrate comprises a polymer hydrogel comprising, within the matrix of said polymer substrate, a biocompatible extracellular matrix protein, a synthetic or engineered matrix polypeptide, or other engineered polypeptide(s). In some embodiments, an engineered matrix polypeptide includes poly-L-lysine, poly-D-lysine, poly-ornithine, vitronectin or erythronectin. In some embodiments, an engineered polypeptide includes domains of extracellular matrix proteins that bind to integrin receptors, domains in extracellular matrix proteins that bind to integrin receptors, and others well known to persons of ordinary skill in the art.

Biocompatible materials useful in the film layers can include non-absorbable polymers such as polypropylene, polyethylene, polyethylene terephthalate, polytetrafluoroethylene, polyaryletherketone, nylon, fluorinated ethylene propylene, polybutester, and silicone, or copolymers thereof (e.g., a copolymer of polypropylene and polyethylene); absorbable polymers such as polyglycolic acid (PGA), polylactic acid (PLA), polycaprolactone, and polyhydroxyalkanoate, or copolymers thereof (e.g., a copolymer of PGA and PLA); or tissue based materials (e.g., collagen or other biological material or tissue obtained from the patient who is to receive the scaffold or obtained from another person.) The polymers can be of the D-isoform, the L-isoform, or a mixture of both.

In some embodiments, other materials can be selected to be used as the substrate material, which can be selected from the group consisting of hydroxyapatite (HAP), tricalcium phosphate (TCP), tetracalcium phosphate (TTCP), dicalcium phosphate anhydrous (DCPA), dicalcium phosphate dihydrate (DCPD), octacalcium phosphate (OCP), calcium pyrophosphate (CPP), collagen, gelatin, hyaluronic acid, chitin, and poly(ethylene glycol). In alternative embodiments, the substrate can also comprise additional material, for example, but are not limited to calcium alginate, agarose, types I, II, IV or other collagen isoform, fibrin, hyaluronate derivatives or other materials.

In some embodiments, the CMS can comprise within or upon the substrate, additional components selected from the group including extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof. Alternatively, or in addition, the structure can comprise at least one conducting polymer selected from poly(pyrrole)s, poly(acetylene)s, poly(thiophene)s, poly(aniline)s, poly(fluorene)s, Poly(3-hexylthiophene), poly naphthalene, poly(p-phenylene sulfide), poly(N-Isopropylacrylamide) (PIPAAm), and poly(para-phenylene vinylene)s. In some cases, the polymer structure comprises an integral pattern of the polymer and molecular remnant traces of poly(N-Isopropylacrylamide).

In some embodiments, the polymer structure composed of or comprising at least one biological hydrogel selected from fibrin, collagen, gelatin, elastin and other protein and/or carbohydrate derived gels or synthetic hydrogel selected from polyethylene glycol, polyvinyl alcohol, polyacrylamide, poly(N-isopropylacrylamide), poly(hydroxyethyl methacrylate) and other synthetic hydrogels, and combinations thereof.

In one embodiment the scaffold material has a plurality of patterned zones. The scaffold material may have a plurality of patterned zones and one or more of the zones in the plurality of zones have a diameter, measured along the longest axis of the zone, of about 100 to about 10,000 μmicrometers. The scaffold material may have a plurality of zones and one or more of the zones of the plurality are essentially square, rectangular, round, oval, sinusoidal, or diamond-shaped.

In one embodiment the thickness of one or more of the films within the scaffold is may be about or less than about 0.25 cm. For example, the scaffold can be formed from two or more films, which can be of the same or different thicknesses. For example, the films can be about or less than about 0.20 cm; about or less than about 0.18 cm; about or less than about 0.16 cm; about or less than about 0.14 cm; about or less than about 0.12 cm; about or less than about 0.10 cm; about or less than about 0.05 cm; about or less than about 0.025 cm; about or less than about 0.020 cm; about or less than about 0.015 cm; about or less than about 0.014 cm; about or less than about 0.013 cm; about or less than about 0.012 cm; about or less than about 0.011 cm; about or less than about 0.010 cm; about or less than about 0.009 cm; about or less than about 0.008 cm; about or less than about 0.007 cm; about or less than about 0.006 cm; about or less than about 0.005 cm; about or less than about 0.004 cm; about or less than about 0.003 cm; about or less than about 0.002 cm; or about 0.001 cm.

In some embodiments, the CMS will be configured using a polymer construction which mimics the rigidity of the tissue, e.g., normal smooth muscle. The rigidity of normal muscle typically varies from 5 kPa to 40 kPa. In some embodiments, the CMS will be configured using a polymer construction which mimics diseased or aged muscle, which typically has a rigidity varying between 30 kPa to 200 kPa. Accordingly, the CMS comprises a polymer substrate that has a rigidity in the range of 5 to 200 kPa, for example, a rigidity of at least about 5 kPa, or at least about 10 kPa, or at least about 20 kPa, or at least about 30 kPa, or at least about 40 kPa, or at least about 50 kPa, or at least about 60 kPa, or at least about 70 kPa, or at least about 80 kPa, or at least about 90 kPa, or at least about 100 kPa, or at least about 120 kPa, or at least about 140 kPa, or at least about 160 kPa, or at least about 180 kPa, or at least about 200 kPa or more than 200 kPa, or any integer between 5-200 kPa.

Thus, although there have been described particular embodiments of the present invention of a new and useful Tissue Scaffold with Patterned Microstructure it is not intended that such references be construed as limitations upon the scope of this invention except as set forth in the following claims.

What is claimed is:

1.-14. (canceled)

15. A tissue scaffold comprising:
a substrate, the substrate further comprising a base layer, the base layer comprising a first pattern of microstructures;
the first pattern of microstructures further comprising at least one first capping layer disposed on the first pattern of microstructures;
the at least one first capping layer comprising a smooth microstructure layer and/or a second pattern of microstructures; and
wherein the first pattern of microstructures and the at least one first capping layer are configured to form a tissue growth surface.

16. The tissue scaffold of claim 15, wherein the smooth microstructure layer and the second pattern of microstructures each have a width of 0.1 μm to 1000 μm.

17. The tissue scaffold of claim 15, wherein the first pattern of microstructures and the at least one first capping layer comprise pillars with a circular, square, triangular, or hexagonal cross-section.

18. The tissue scaffold of claim 15, wherein the at least one first capping layer and the first pattern of microstructures are disposed hierarchically on the base layer, wherein the at least one first capping layer and the base layer have different surface energy.

19. The tissue scaffold of claim 18, wherein the first microstructure layer, comprising the at least one first capping layer and the first pattern of microstructures, comprises a water sessile drop contact angle being 100 degrees or less.

20. The tissue scaffold of claim 15, further comprising a second pattern of microstructures, wherein the second pattern of microstructures further comprise at least one second capping layer disposed on the second pattern of microstructures.

21. The tissue scaffold of claim 20, wherein the second microstructure layer, comprising the at least one second capping layer and the second pattern of microstructures, comprises a water sessile drop contact angle greater than 100 degrees.

22. The tissue scaffold of claim 15, further comprising a plurality of cells deposited on the tissue growth surface.

23. The tissue scaffold of claim 22, wherein the plurality of cells are selected from cell types comprising endothelial cells, smooth muscle cells, fibroblasts, tendon cells, mesenchymal stem cells, skeletal muscle cells, chondrocytes, and epithelial cells.

24. The tissue scaffold of claim 23, wherein the endothelial cells are vascular endothelial cells, the smooth muscle cells are vascular smooth muscle cells, and the mesenchymal stem cells are bone marrow-derived human mesenchymal stem cells.

25. The tissue scaffold of claim 15, wherein an avoidance layer is deposited on all or a portion of the at least one first capping layer.

26. A tissue scaffold comprising:
a substrate, the substrate further comprising a base layer, the base layer comprising an at least two hierarchical microstructure patterns;
the at least two hierarchical microstructure patterns comprising at least one first pattern of microstructures and an at least one second pattern of microstructures;
the at least one first pattern of microstructures further comprising at least one first capping layer;
the at least one second pattern of microstructures further comprising at least one second capping layer;
the at least one first capping layer being hierarchically disposed on the at least one first pattern of microstructures;
the at least one second capping layer being hierarchically disposed on the at least one second pattern of microstructures;
wherein the at least two hierarchical microstructure patterns are disposed of in a geometric pattern on the substrate, wherein the geometric pattern comprises a surface energy pattern; and
the surface energy pattern directs at least one cell type in contact with at least one of the at least two hierarchical microstructure patterns to form at least one tissue structure.

27. The tissue scaffold of claim 26, wherein the at least one tissue structure is of a size at least ten times the size of the constituent of the at least one cell type.

28. The tissue scaffold of claim 27, wherein the at least one cell type forms a confluent monolayer across an extracellular matrix while maintaining the surface energy pattern, the surface energy pattern further comprising alternating at least one high surface energy microstructure and at least one low surface energy microstructure.

29. The tissue scaffold of claim 26, wherein the at least two hierarchical microstructure patterns further comprise ridges and/or grooves.

30. The tissue scaffold of claim 29, wherein the ridges and the grooves have a same geometry or a variety of geometries, the same geometry and variety of geometries comprising convex, concave, or substantially planar surfaces.

31. A method of manufacturing a patterned tissue scaffold, comprising the steps of:
applying a photoresist to a base layer to produce a photoresist-coated base layer;
covering the photoresist-coated base layer with a photomask characterized by a microstructure pattern to produce a masked ensemble;
exposing the masked ensemble to a UV radiation to create an exposed ensemble;
removing the photomask from the exposed ensemble to provide a photoresist-coated base layer comprising a UV-exposed microstructure pattern;
developing said photoresist-coated base layer to remove the UV-exposed portions of photoresist providing an exposed surface of the base layer characterized by a pattern corresponding to the microstructure pattern of the photomask;
applying a polymer to the exposed surface of the base layer; and
removing residual photoresist to reveal a pattern polymer layer microstructure on the base layer to provide a pattern polymer layer microstructure corresponding to the microstructure pattern of the photomask.

32. The method of claim 31, wherein spin coating is used for applying the photoresist to the base layer.

33. The method of claim 31, further comprising printing polymers on a transitional polymer, wherein printing multiple polymer structures comprising different surface energies creates stacked printings.

34. The method of claim 31, wherein binding the polymer occurs by a hydrophilic force, hydrophobic force, ionic force, covalent force, Van der Waals force, hydrogen bonding, and/or physical entanglement, and combinations thereof.

35. The method of claim 29, wherein applying the photoresist to the base utilizes a combination of positive and negative photoresists.
